(19) United States
(12) Patent Application Publication
(10) Pub. No.: US 2026/0335658 A1
(43) Pub. Date: Oct. 29, 2026

(54) BRAIN NERVE ELECTRICAL STIMULATION SYSTEM WITH SENSING FUNCTION

(71) Applicant: JIANGSU CED MEDTECH CO., LTD, Nantong (CN)

(72) Inventors: Rongqing DAI, Nantong (CN); Grace Ying Yang JANG, Nantong (CN)

(73) Assignee: JIANGSU CED MEDTECH CO., LTD, Nantong (CN)

(21) Appl. No.: 19/102,508

(22) PCT Filed: Aug. 31, 2023
(86) PCT No.: PCT/CN2023/116021

§ 371 Date: Feb. 10, 2025

(30) Foreign Application Priority Data
Aug. 8, 2022 (CN) 202210943820.9

(51) Int. Cl. A61N 1/372; A61N 1/05; A61N 1/36; A61N 1/378

(52) U.S. Cl. CPC A61N 1/37252; A61N 1/0529; A61N 1/36128; A61N 1/3787

(57) ABSTRACT

Disclosed in the present invention is a brain nerve electrical stimulation system with a sensing function. The brain nerve electrical stimulation system comprises an implant stimulator, a stimulation electrode, a multifunctional headgear, a remote controller, and a program controller. The implant stimulator comprises an implant main body and a first coil, with the first coil arranged outside the implant main body. The implant main body comprises an electronics enclosure sealed by a metal shell and an electrode connector. A first power supply and a circuit mainboard are arranged in the electronics enclosure, and a current pulse generator is arranged on the circuit mainboard. A current pulse output channel of the current pulse generator is connected to the stimulation electrode by means of the electrode connector. The multifunctional headgear comprises a second coil, and the second coil and the first coil engage in inductive coupling to charge the first power supply and transmit a data signal. The brain nerve electrical stimulation system removes an electrode wire extension line, thus reducing the volume of the implant stimulator and minimizing surgical trauma. Simultaneously, the system is equipped with a multifunctional headgear, eliminating the need for extra fixing measures, which allows for rapid charging as well as safe data communication, thereby improving the treatment experience of a patient.

### TECHNICAL FIELD

The present invention relates to the field of medical instruments, and more particularly to a brain nerve electrical stimulation system with a sensing function.

### BACKGROUND ART

At present, a clinical brain nerve electrical stimulation implant, i.e., a deep brain stimulator (DBS), can only be implanted in the chest due to its large size, which requires a neurosurgeon to spend nearly 4 hours making a subcutaneous tunnel to wire an electrode extension cable from the top of the brain to the chest in order to connect the brain pacemaker. In the clinical use by a patient implanted with the brain pacemaker, two electrode extension cables with a length of about 40 to 50 cm extending from the top of brain to the chest are the most vulnerable part of the implanted components, because the head and the neck of a human will rotate numerous times per day. Fracture of the electrode extension cable is a common clinical adverse event. In addition, infection caused by the wires extending from the head to the chest is another type of commonly occurring and safety-critical adverse event, depending on the patient's health condition.

A patent of Beijing Pins Medical Co. Ltd. with patent No. CN103768712B describes a head-implanted deep brain stimulation system. In this invention, a small-capacity rechargeable battery is used as the pulse generator (IPG) of the brain pacemaker, which can be directly implanted on the skull, simplifying the implantation surgery and reducing the risk of wire fracture or infection caused by long electrode wires. However, since the control circuit of its IPG is composed of discrete components and the charging coil is placed inside the device, the physical size of the IPG can not be minimized, and the charging efficiency is affected by the internally placed charging coil, which poses a challenge to the limit the temperature rise of the IPG case during charging. Although the patent mentions the possible configuration of an external charging coil, different antennas are still used for charging and data communication, the IPG size is still limited and a considerable craniotomy size is required for implantation.

A patent CN108290045A filed by Boston Scientific Corporation in China discloses a trans-cranially mounted brain pacemaker whose volume can be made smaller in principle; however, its IPG does not include a battery, requiring an external support device to supply power all the time, which limited the extent of its clinical application; in addition, it uses an externally hung electrode connector, which complicates the implant surgical procedure and increase the risk of infection due to the size of the electrode connector.

In addition, trans-cranially mounted IPG devices are susceptible to impact damage when the head strikes hard objects, neither of the IPG fixation methods of the two above inventions provides such protection. Functionally, neither IPG includes feedback functions for neural or other signals and therefore does not offer the possibility of closed-loop stimulation control.

### SUMMARY OF THE INVENTION

A technical problem to be solved by the present invention is to provide a brain nerve electrical stimulation system with a sensing function, which simplifies the structure of an implant stimulator, reduces the volume of the implant stimulator and reduces surgical trauma, and at the same time is equipped with an external control apparatus that is easy to be worn, can be charged quickly and can perform secure data communication and improve treatment experience.

In order to solve the above technical problem, the present invention provides a brain nerve electrical stimulation system with a sensing function, which includes a cranial-mounted implant stimulator with a stimulation electrode, a conveniently wearable multifunctional headgear, a patient remote controller and a clinician stimulation program controller; the implant stimulator includes an implant main body and a first coil, the implant main body includes an electronics enclosure sealed by a metal shell and an electrode connector encapsulated by a non-metal material, the first coil is installed outside the metal shell, a first power supply and a circuit mainboard are arranged inside the electronics enclosure, and a current pulse generator is arranged on the circuit mainboard; the pulse output channels of the current pulse generator are connected to the stimulation electrode by means of the electrode connector to realize electrical stimulation function; the metal shell serves as a return electrode for current pulses; the multifunctional headgear includes a second coil, a second rechargeable battery and a controller, where the second coil, the second power supply and the controller are all sewn into a headgear fabric to be comfortably worn by the patient, and when the headgear is worn by the patient, the second coil and the first coil charge the first power supply through inductive coupling and perform radio frequency data communication as required; the clinician stimulation program controller communicates with the multifunctional headgear via Bluetooth, setting up stimulation prescriptions for the implant stimulator, detecting and analyzing the operating condition of the implant stimulator, and exchanging data with a cloud server; the stimulation prescription includes stimulating electrode contacts, stimulation mode, pulse width, pulse frequency, or/and, adjustable pulse amplitude range; the patient remote controller is used by the patient to control over the implant stimulator, including starting or ending stimulation, adjusting stimulation intensity, inputting medication state, or/and starting or ending a charging process.

Preferably, the 2n independent current pulse generators are arranged on the circuit mainboard to constitute 2n independently controllable current pulse stimulation channels, 2n annular electrode contacts are connected to the stimulation channels through the electrode connector, each of the current pulse stimulation channels of the stimulator in the hermetic electronics enclosure is connected to a corresponding contact of the electrode connector via pins of a feedthrough connector, and the electrode connector constitutes two n-channel connection sockets to be connected to all electrode wires of the two stimulation electrodes, respectively, each stimulation electrode containing n stimulation contacts, where n is a positive integer no less than 4.

Preferably, a conductor of the first coil is a cable formed by twisting a plurality of pure gold wires, where the first coil is a flat coil with a protective sleeve and is spirally and horizontally wound with the cable and injection-molded from a biocompatible flexible material and has a thickness of less than 3 mm, and is integrally encapsulated with the metal shell by the flexible material; an output end of the first coil is connected to a charging module and a data communication module on the circuit mainboard in the hermetic electronics enclosure via the pins of the feedthrough connector, and when implanted into the human body, the first coil can be directly placed between the scalp and the skull, without cutting the skull open, and serves to fix the implant main body.

Preferably, the first coil further includes a Bluetooth communication component in the protective sleeve, the Bluetooth communication component is a Bluetooth chip antenna and a ceramic circuit board component carrying the Bluetooth chip antenna, or the Bluetooth communication component is a wire antenna wound in a predetermined shape or a predetermined length, the Bluetooth communication component is encapsulated with an insulating material and then encapsulated with the flexible material together with the first coil, and is connected to the first Bluetooth transceiver of the circuit mainboard via the pins of the feedthrough connector.

Preferably, the first coil and the electrode connector are installed in the same side of the metal shell, the electrode connector and the protective sleeve of the first coil are integrally formed and then fixed onto a side wall of the metal shell, and an output end of the first coil is connected to the charging module and the data communication module on the circuit mainboard via the pins of the feedthrough connector.

Preferably, the first coil and the electrode connector are separately installed on two sides of the metal shell, the electrode connector is fixed on one side wall of the metal shell, the protective sleeve of the first coil is fixed on the other side wall of the metal shell, and when the implant stimulator is implanted into the human body, the implant main body is directly implanted on the skull without perforating the skull.

Preferably, the electrode connector includes a connection socket and a connector shell arranged outside the connection socket, the connection socket including a retaining member for securing the stimulation electrode and an annular electrode contact included of a plurality of metal rings and metal spring rings located in the metal rings, an insulating ring provided between two adjacent metal rings, the retaining member having an electrode protective sleeve provided at an end thereof.

Preferably, the electrode connector is directly adhered to the side wall of the metal shell, or a fixed block is provided on the side wall of the metal shell connected to the electrode connector, and the electrode connector is connected to the side wall of the metal shell through the fixed block when it is encapsulated with epoxy resin or polyurethane (PU).

Preferably, the first power supply is a first rechargeable battery, the metal shell includes a first shell and a second shell which are tightly fitted together, the second shell is one bottom board, a protruding strip is provided on the bottom board, the first rechargeable battery is placed within enclosure of the protruding strip, the circuit mainboard is installed on top of the first rechargeable battery, and insulating material is provided at the contact places between the bottom board, the first rechargeable battery and the circuit mainboard for electrical isolation; the first shell is box-shaped and is snap-fit onto the second shell; a through hole matching the feedthrough connector is provided on the side wall of the second shell connected to the first coil and the electrode connector; the feedthrough connectors on two sides are respectively mounted in the corresponding through holes, and second fixed hanging plates are respectively provided on the other two side walls of the second shell.

Preferably, the first power supply is a first rechargeable battery, an ASIC chip, a first microprocessor, a first Bluetooth transceiver and an inertial measurement processor are arranged on the circuit mainboard, the current pulse generator is provided in the ASIC chip, and the ASIC chip further includes a neural signal detection module, a wireless charging management module, a bidirectional radio frequency data communication module and a first power management module, the first Bluetooth transceiver, the inertial measurement processor and the current pulse generator, the nerve signal detection module and the bidirectional radio frequency data communication module are all connected to the first microprocessor interface; the first microprocessor controls input electrode selection, signal gain and frequency response range of the neural signal detection module, charging current and charging flow output by the wireless charging management module, and supply voltage, stimulation current, pulse width and pulse frequency of the current pulse generator.

Preferably, the first microprocessor sends a stimulation unit data packet containing a stimulation mode and a stimulation amplitude by instruction to a stimulation unit control circuit of the current pulse generator, the stimulation unit control circuit transferring a stimulation amplitude value to a digital-to-analog converter of the current pulse generator and converting same into a current value and then into an amplitude of an output pulse of the current pulse generator via an electrode drive circuit.

Preferably, the stimulation unit control circuit transfers stimulation mode information of the stimulation unit data packet to switching logic that controls switching of cathode and anode current output switches and return electrodes of the electrode driver by a level shifter to control pulse width, pulse frequency, output polarity and output mode of current stimulation pulses via input control signals.

Preferably, the number of stimulation electrodes is two, and the stimulation electrodes can be used for stimulating areas of two brain hemispheres, respectively, each electrode containing at least four stimulation contacts; the neural signal detection module combines any of two or more stimulation electrode contacts as measuring electrode inputs for detecting potential of the nerve area, and the measured signals are stored in internal memory of the implant as required or fed back to the multifunctional headgear via the bidirectional radio frequency data communication module and the first coil.

Preferably, the inertial measurement processor detects patient motion and cardiac rhythm changes and feeds back the measured signal to the multifunctional headgear via the bidirectional radio frequency data communication module and the first coil.

Preferably, the ASIC chip, the microprocessor, the first Bluetooth transceiver, and the inertial measurement processor are arranged in the same side of the circuit mainboard and the first rechargeable battery is placed on the other side of the circuit mainboard. Preferably, the metal shell includes a first shell and a second shell which are tightly fitted, a pressure-sensitive adhesive sheet, a frame, the first rechargeable battery, a rubber gasket and the circuit mainboard are successively provided between the first shell and the second shell, the frame is tightly fitted to the circuit mainboard, the first rechargeable battery is provided in the frame, and a protective solder strip is provided on a periphery of the frame.

Preferably, a PCB mounting board is vertically connected to a side of the circuit mainboard, the frame is divided into a first space and a second space, the first rechargeable battery is placed in the first space, an end side of the second space is an open end, and a feedthrough connector is matched and mounted, the second space is provided with bumps along side walls of the two sides, slots are provided on the bumps, the two ends of the PCB mounting board are inserted into the slots for fixing, and the pins of the feedthrough connector placed on the end side of the second space are connected to the mounting board.

Preferably, the metal shell includes a first shell and a second shell which are tightly fitted, the first rechargeable battery and the circuit mainboard are placed between the first shell and the second shell, the circuit mainboard is a rigid-flexible circuit mainboard and is bent to surround the first rechargeable battery.

The present invention further includes a mounting bracket including a base plate, a cover plate and screws, lugs are provided at edges of the base plate and the cover plate, where threaded holes are provided on the lugs, the screws can fix the base plate to an edge of an opening of the skull via the threaded holes, the implant main body is placed on the base plate, the cover plate covers the top of the implant main body, and the screws can fix the cover plate to the edge of the opening of the skull via the threaded holes on the cover plate, a surface of the implant main body with the cover plate mounted is substantially flush with a surface at the first coil.

Preferably, the first shell is provided with a plurality of first fixed hanging plates, and the first fixed hanging plates are provided with third threaded holes.

Preferably, the second coil is a flat coil spirally wound from a twisted cable twisted from a plurality of insulated wires, or from a single wire, the periphery of the second coil being injection molded from a flexible material, encapsulated and then sewn in place within the fabric of the multifunctional headgear, the multifunctional headgear being a cap conforming to the skull contour of a patient, the multifunctional headgear being worn with the second coil covering the first coil.

Preferably, the second power supply is a second rechargeable battery, and the controller is connected to the second rechargeable battery and the controller is connected to the second coil via a flexible cable, respectively.

Preferably, the controller includes a second microprocessor, a second Bluetooth transceiver, a second power management module and a coil drive module, the second microprocessor includes a signal processing module, a stimulation adjustment processing module and a charging control firmware functional module respectively used for processing and analyzing signals fed back by the implant stimulator, adjusting stimulation parameters according to the feedback signals and managing the charging process of the multifunctional headgear on the implant; the coil drive module includes a coil drive circuit, a radio frequency signal transceiver circuit and a coil induction charging control interface circuit; the second power management module includes a charging circuit of a second rechargeable battery, a charging input interface, a battery protection circuit and a voltage regulation circuit; and the second Bluetooth transceiver provides a Bluetooth communication channel of a multifunctional headgear.

Preferably, the second coil is sewn into an insulating cap fabric corresponding to the first coil position of the implant, the controller is a thin rigid-flexible circuit mainboard encapsulated by epoxy resin and sewn into the fabric of one side edge of the multifunctional headgear, and the second rechargeable battery is sewn into the other side edge of the multifunctional headgear.

Preferably, the controller is provided with a radio frequency signal strength detection circuit for determining and reminding whether the position of the multifunctional headgear is shifted.

Preferably, the doctor sets up and adjusts the stimulation prescription using the program controller for the implant stimulator via Bluetooth communication with the multifunctional headgear and the patient performs daily operations using the remote controller on the implant stimulator via Bluetooth communication.

The present invention has the following advantageous effects compared with the prior art: 1. The cranial-mounted implant stimulator provided by the present invention allows the implant to be fixed on the skull, greatly reducing the complexity of the implantation surgery, eliminating infection risks and failure risks caused by the electrode wire extension line, overcoming the difficulties in current clinical practice, eliminating the extra hours of surgery time for the doctor to tunnel the wire to the chest in order to connect with the brain pacemaker, as well as the infection risk and failure risk of the electrode extension cable during clinical treatment. 2. The implant stimulator provided by the present invention has a compact and reasonable internal structure, the total thickness can be controlled within 6.5 mm, only about 25 mm*25 mm skull grinding is required, the skull is not required to be completely opened, even the skull is not required to be perforated, there is no dural contact risk, and the surgical trauma is minimized. 3. The flexible first coil in the implant stimulator and the flexible second coil in the multifunctional headgear of the present invention can achieve close inductive coupling, which can effectively improve the charging efficiency of the implant stimulator battery and reduce the risk of temperature rise due to the high RF power consumption required for charging the built-in coil in the shell. 4. The flexible first coil of the implant stimulator provided by the present invention also serves as a radio frequency data antenna, effectively simplifying the physical structure of the implant stimulator and reducing the volume of the implant stimulator. 5. The short-range radio-frequency data communication of centimeter level realized by the charging coil provided by the present invention effectively improves the security of data, and can be used in combination with the Bluetooth communication channel simultaneously equipped with the multifunctional headgear to provide the possibility of dual-channel authentication for the charging of implant and remote override, etc. 6. Clinical trials have demonstrated that the closed-loop stimulation mode of Deep Brain Stimulation Therapy (DBS) and (Reactive Nerve Stimulation (RNS) is significant to the therapy effect. The present invention provides a novel real-time closed-loop stimulation mode that is implemented by the cooperation of an implant and a multifunctional headgear. Compared with the closed-loop control independently realized by the implant stimulator, the method has more flexible characteristic, is not limited by the power consumption of the implant, and can realize algorithm adjustment more conveniently. 7. The implant stimulator and multifunctional headgear formation measurement, control and stimulation system provided by the present invention provides a natural and effective research platform for clinical research and brain-instrument interface development. 8. The present invention provides a mounting bracket structure for an implant stimulator that further simplifies device implantation surgery and provides implant protection against the risk of impact.

### BRIEF DESCRIPTION OF THE DRAWINGS

FIG. 1 is a schematic overall structural diagram showing a brain nerve electrical stimulation system according to an embodiment of the present invention;

FIG. 2 is a schematic diagram showing a frame of a brain nerve electrical stimulation system according to an embodiment of the present invention;

FIG. 3a is a schematic overall structural diagram showing an implant stimulator according to a first embodiment of the present invention, and FIG. 3b is an exploded schematic diagram showing an implant stimulator according to a first embodiment of the present invention;

FIGS. 4a and 4b are schematic structural diagrams showing an internal structure of an implant stimulator according to a first embodiment of the present invention;

FIG. 5 is a schematic circuit diagram showing an implant stimulator according to an embodiment of the present invention;

FIG. 6 is a schematic circuit diagram showing a stimulator pulse generator according to an embodiment of the present invention;

FIG. 7(a) is an internal assembly view of a metal shell according to a first embodiment of the present invention, and FIG. 7(b) is an internal assembly view of a circuit mainboard mounted in a metal shell according to an embodiment of the present invention;

FIG. 8(a) is an internal assembly view of a metal shell according to a second embodiment of the present invention, and FIG. 8(b) is an internal assembly view of a circuit mainboard mounted in a metal shell according to a second embodiment of the present invention;

FIG. 9(a) and 9(d) are a top view, a side view, a front view and a solid diagram, respectively, of a circuit mainboard according to a second embodiment of the present invention;

FIG. 10(a), FIG. 10(b), FIG. 10(c) and FIG. 10(d) are a side view, a top view, a solid diagram and a front view, respectively, of a base plate of an implant mounting bracket according to a first embodiment of the present invention;

FIG. 11(a), FIG. 11(b), FIG. 11(c) and FIG. 11(d) are a side view, a top view, a solid diagram and a front view, respectively, of an upper cover of an implant mounting bracket according to a first embodiment of the present invention;

FIG. 12(a) is a schematic diagram showing a base plate of a mounting bracket on the skull according to a first embodiment of the present invention, FIG. 12(b) and FIG. 12(c) are schematic diagrams showing an implant stimulator inserted between a skull and a scalp and an implant main body placed in the base plate according to a first embodiment of the present invention, and FIG. 12(d) is a schematic diagram showing a cover plate covered on a base plate according to the first embodiment of the present invention;

FIG. 13(a) is a schematic diagram showing a state before an implant stimulator is put into a skull scalp suture according to a first embodiment of the present invention, FIG. 13(b) is a schematic perspective view taken along a direction A-A after the implant stimulator is mounted in FIG. 13(a), and FIG. 13(c) is a schematic perspective view taken along a direction B-B after an implant stimulator is mounted in FIG. 13(a);

FIG. 14(a) is a schematic overall structural diagram showing an implant stimulator according to a third embodiment of the present invention, and FIG. 14(b) is an exploded schematic diagram showing an implant stimulator according to a third embodiment of the present invention;

FIG. 15(a) is a schematic overall structural diagram showing an implant stimulator according to a fourth embodiment of the present invention, FIG. 15(b) is an exploded schematic diagram showing an implant stimulator according to a fourth embodiment of the present invention, and FIG. 15(c) is a schematic structural diagram showing a metal shell according to a fourth embodiment of the present invention;

FIG. 16(a) is a schematic cross-sectional transverse perspective view after mounting an implant stimulator according to a fourth embodiment of the present invention, and

FIG. 16(b) is a schematic longitudinal perspective view after mounting of an implant stimulator according to a fourth embodiment of the present invention;

FIG. 17 is a schematic structural diagram showing a multifunctional headgear according to an embodiment of the present invention;

FIG. 18 is a schematic diagram showing a circuit of a multifunctional headgear according to an embodiment of the present invention;

FIG. 19 is a schematic diagram showing an autonomic mode of a brain nerve electrical stimulation system according to an embodiment of the present invention; and

FIG. 20 is a schematic diagram showing a closed-loop mode of a brain nerve electrical stimulation system according to an embodiment of the present invention.

In the drawings:

1—implant stimulator, 11—implant main body, 111—metal shell, 1111—first shell, 1112—second shell, 1113—third through hole, 1114—protrusion, 1115—fixed block, 112—circuit mainboard, 1121—mounting board, 1122—first through hole, 1123—first side, 1124—second side, 1125—third side, 1126—second through hole, 113—rubber gasket, 114—first rechargeable battery, 1141—anode/cathode, 115—frame, 1151—first space, 1152—second space, 1153—fixed column, 1154—bump, 1155—slot, 116—protective solder strip, 117—pressure—sensitive adhesive sheet, 118—electrode connector, 1181—connection socket, 1181a—retaining member, 1182—electrode contact, 1183—screw, 1184—sealing member, 1185—electrode protective sleeve, 1186—connector shell, 1187—fourth through hole, 1188—fifth through hole, 1189—fourth threaded hole, 12—first coil, 121—protective sleeve, 122—Bluetooth chip antenna, 123—ceramic circuit board, 13—stimulation electrode, 131—electrode skull cap, 15, 151, 152—feedthrough connector, 2—multifunctional headgear, 21—second coil, 22—controller, 23—second rechargeable battery, 24—flexible cable, 3—remote controller, 4—program controller, 5—skull, 6—mounting bracket, 61—base plate, 62—first hanger, 621—first threaded hole, 63—screw, 64—cover plate, 65—second hanger, 651—second threaded hole, 7—first fixed hanging plate, 71—third threaded hole, 8—scalp, 9—cranial dura, 10—second fixed hanging plate.

### DETAILED DESCRIPTION OF THE INVENTION

The invention will now be further described referring to the accompanying drawings and examples.

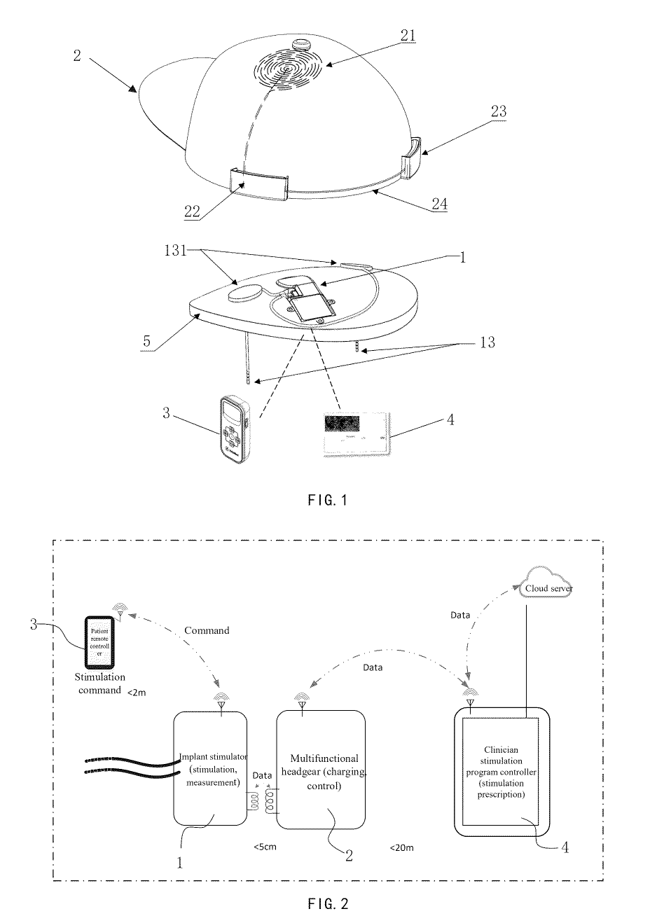

FIG. 1 is a schematic overall structural diagram showing a brain nerve electrical stimulation system according to an embodiment of the present invention; and FIG. 2 is a schematic diagram showing a frame of a brain nerve electrical stimulation system according to an embodiment of the present invention.

Referring to FIGS. 1, 2 and 3b, the present invention provides a brain nerve electrical stimulation system with a sensing function, which includes a cranial-mounted implant stimulator 1, a conveniently wearable multifunctional headgear 2, a patient remote controller 3 and a clinician stimulation program controller 4, and the multifunctional headgear is preferably a cap conforming to the patient's skull and can be worn on the patient's head. The implant stimulator 1 includes an implant main body 11, a stimulation electrode 13 and a first coil 12; the implant main body 11 includes an electronics enclosure sealed by a metal shell 111 and an electrode connector 118 encapsulated by a non-metal material; the first coil 12 is installed outside the metal shell 111; a first power supply and a circuit mainboard 112 are installed inside the electronics enclosure; the circuit mainboard 112 is connected to the electrode connector 118; the electrode connector 118 is connected to the stimulation electrode 13; and a current pulse generator is installed on the circuit mainboard 112; the pulse output channels of the current pulse generator are connected to the stimulation electrode 13 by means of the electrode connector 118 to realize electrical stimulation function; the metal shell 111 acts as a return electrode for the current pulses. The multifunctional headgear 2 includes a second coil 21, a second power supply and a controller 22; in the present embodiment, the second power supply is a second rechargeable battery 23; the second coil 21, the second rechargeable battery 23 and the controller 22 are all sewn into a headgear fabric to be comfortably worn by the patient; and when the headgear is worn by the patient, the second coil 21 and the first coil 12 achieve tight inductive coupling to charge the first power supply and perform radio frequency data communication. In the present embodiment, the first power supply is a first rechargeable battery 114, preferably a rechargeable lithium battery. The clinician stimulation program controller 4 communicates with the multifunctional headgear 2 via Bluetooth, setting up a stimulation prescriptions for the implant stimulator 1, detecting and analyzing the operating condition of the implant stimulator 1, and exchanging data with the cloud server; the stimulation prescription includes parameters such as stimulating electrode contacts, stimulation mode, pulse width, pulse frequency, and adjustable pulse amplitude range; the patient remote controller 3 is used to by the patient to exercise basic operation control over the implant stimulator 1, including starting or ending stimulation, adjusting stimulation intensity, inputting medication state, and starting or ending the charging process, etc.

The embodiments of the present invention provide a brain nerve electrical stimulation system including a cranial-mounted implant stimulator 1 and a multifunctional headgear 2 suitable for long-term wear, which together with a clinician stimulation program controller 4 used by a doctor and a remote controller 3 used by a patient constitute a novel brain nerve electrical stimulation system. The functional surgery of the brain nerve electrical stimulation system is as shown in FIG. 2: in the first embodiment, referring to FIGS. 3b, 4a and 4b, 2n independent current pulse generators are arranged on the circuit mainboard 112 of the implant stimulator 1, where n is a positive integer not less than 4 and 2n is an integer of eight or more, so as to constitute eight or more independently controllable current pulse stimulation channels; at least eight annular electrode contacts 1182 are connected to the stimulation channels through the electrode connector 118, where the annular electrode contacts 1182 are composed of a metal ring and a metal spring ring located in the metal ring, and an insulating ring is provided between each adjacent two metal rings; each current pulse stimulation channel in the hermetic electronics enclosure is connected by a pin of the feedthrough connector 15 to a corresponding contact of an electrode connector 118, which constitutes a connection socket for two n-channels and is connected to all electrode wires of two stimulation electrodes 13 respectively, each stimulation electrode 13 including at least four stimulation contacts. Two at least four-contact stimulation electrodes support dual-chamber deep brain stimulation, and each electrode stimulation contact simultaneously supports the application of neural signal detection. In other embodiments, a greater number of current pulse channels may be provided, and a corresponding greater number of electrode contacts 1182 may be provided in the electrode connector 118, such as 16 electrode contacts 1182, and 8 electrode contacts on each side of the electrode connector 118 to form two 8-channel connection sockets, and so on, or a greater number of electrode contacts 1182, such as 32 electrode contacts 1182, as long as the electrode connector 118 is appropriate in size. The implant stimulator 1 uses a small rechargeable battery, can independently support continuous stimulation at a nominal current intensity for more than 3 days, and can be rapidly charged through the first coil 12 and the multifunctional headgear 2. The implant stimulator 1 can also use a built-in neural signal detection module and an inertial measurement processor to realize real-time closed-loop stimulation function together with the multifunctional headgear 2 through radio frequency data connection. Meanwhile, a clinician can adjust the stimulation prescription of the implant stimulator 1 together with the multifunctional headgear 2 through Bluetooth communication using a clinician stimulation program controller 4, and a patient can select the stimulation prescription and adjust the stimulation intensity through Bluetooth communication using the remote controller 3, etc.

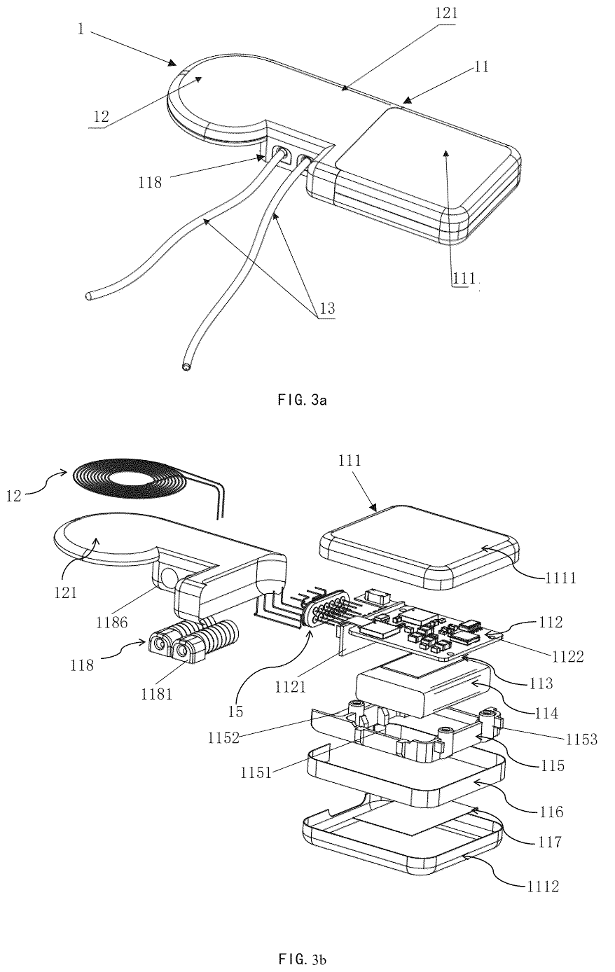

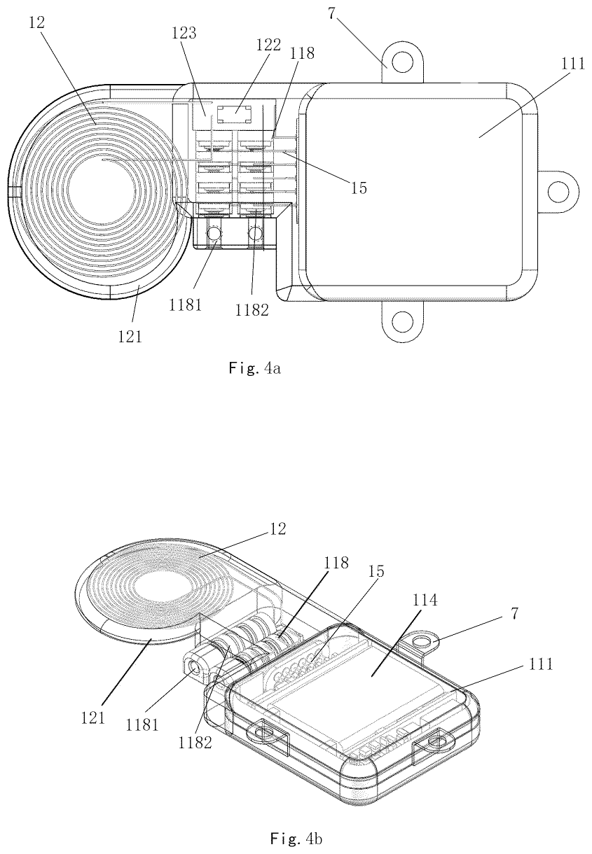

FIG. 3a is a schematic overall structural diagram showing an implant stimulator according to a first embodiment of the present invention, FIG. 3b is an exploded view showing an implant stimulator according to the first embodiment of the present invention, and FIGS. 4a and 4b are schematic inner structural diagrams showing an implant stimulator according to the first embodiment of the present invention. Referring to FIGS. 3a and 4b, in the first embodiment, the implant stimulator 1 is composed of an implant main body 11, two stimulation electrodes 13 and a first coil 12, where the implant main body 11 includes an electronic part enclosure sealed by a titanium metal shell 111 and an electrode connector 118 encapsulated by PU (polyurethane) injection molding or epoxy resin, the hermetic electronics enclosure houses a medical first rechargeable battery 114 and a circuit mainboard 112, and the titanium metal shell 111 is used as a stimulation return electrode in a monopolar stimulation mode. Further, the first coil 12 is arranged outside the metal shell 111, the conductor of the first coil 12 is a cable formed by twisting a plurality of pure gold wires, and the first coil 12 is a flexible flat coil with a protective sleeve 121, which is spirally and horizontally wound with the cable and is injection-molded from a biocompatible flexible material with a thickness of less than 3 mm, and is encapsulated in one piece with the metal shell 111 with a flexible material, where the flexible material is preferably silica gel or polyurethane (PU); an output end of the first coil 12 is connected to the charging module and the data communication module on the circuit mainboard 112 in the hermetic electronics enclosure via pins of a feedthrough connector 15, and when implanted into a human body, the first coil 12 can be directly placed between the scalp and the skull, without the need for placement by craniotomy, i.e., without cutting the skull open, thereby reducing the craniotomy area and the skull surgical wound, and serving to fix the implant main body 11. Further, a Bluetooth communication component is provided in the protective sleeve 121 outside the first coil 12, and the Bluetooth communication component includes a Bluetooth chip antenna 122 and a ceramic circuit board 123 carrying the Bluetooth chip antenna 122, where the Bluetooth communication component is encapsulated with epoxy or other insulating material and then encapsulated with a flexible material such as silicone or polyurethane together with the first coil 12, and is connected to a first Bluetooth transceiver of the circuit mainboard 112 via pins of the feedthrough connector 15.

Further, in the first embodiment, the first coil 12 and the electrode connector 118 are installed onto a same side of the metal shell 111, and the electrode connector 118 and the protective sleeve 121 of the first coil 12 are integrally formed and fixed to a side wall of the metal shell 111.

The first coil 12 is arranged outside the metal shell 111 and is provided as a thin coil encapsulated by a biocompatible flexible material such as silicone or polyurethane, and the following effects are achieved: (1) With respect to a structure in which a charging coil is provided in the metal shell, a volume of the implant stimulator is reduced so as to reduce the requirement for craniotomy area; (2) It is easy to mount, can be placed directly between the skull and scalp, without occupying craniotomy space, so as to reduce a surgical wound of the skull; (3) When implanted into the human body, it can play a role of fixing the implant main body, (4) It improves charging efficiency and reduce the risk of heating emitted from the shell at the time of charging, and (5) The first coil is used as an inductively coupled data transmission antenna at the same time to realize centimeter-level near-distance data transmission and improve the secrecy performance of data communication. (6) It reduces attenuation of the communication signal solves the problem that the high-frequency signal cannot be transmitted in the metal shell, and the low-frequency transmission will increase the antenna volume, which is not conducive to the implanted product.

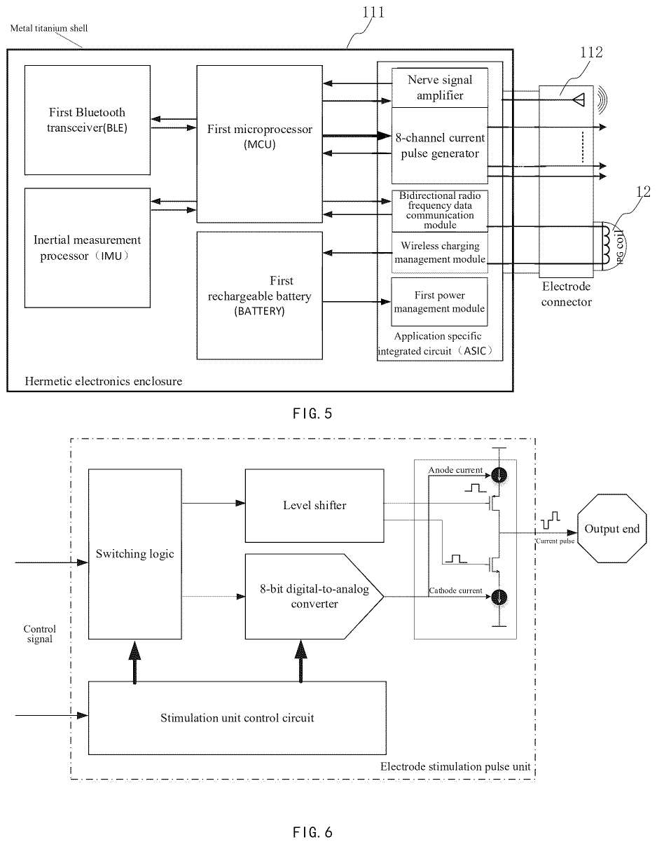

FIG. 5 is a schematic circuit diagram showing an implant stimulator according to an embodiment of the present invention; FIG. 6 is a schematic circuit diagram showing a stimulator pulse generator according to an embodiment of the present invention.

Referring to FIG. 5, an ASIC chip, a first microprocessor (MCU), a first Bluetooth transceiver (BLE) and an inertial measurement processor (IMU) are arranged on the circuit mainboard 112, the current pulse generator is provided in the ASIC chip, and the ASIC chip further includes a neural signal detection module, a wireless charging management module, a bidirectional radio frequency data communication module and a first power management module, the first Bluetooth transceiver, the inertial measurement processor and the current pulse generator, the neural signal detection module and the bidirectional radio frequency data communication module are all connected to the first microprocessor interface; the first microprocessor controls input electrode selection, signal gain and frequency response range of the neural signal detection module, charging current and charging flow output by the wireless charging management module, and supply voltage, stimulation current, pulse width and pulse frequency of the current pulse generator.

Referring to FIG. 6, the first microprocessor sends a stimulation unit data packet containing a stimulation mode and a stimulation amplitude by instruction to a stimulation unit control circuit of the current pulse generator, the stimulation unit control circuit transferring a stimulation amplitude value to a digital-to-analog converter of the current pulse generator and converting same into a current value and then into an amplitude of an output pulse of the current pulse generator via an electrode drive circuit. Meanwhile, the stimulation unit control circuit transfers stimulation mode information of the stimulation unit data packet to switching logic that controls switching of cathode and anode current output switches and return electrodes of the electrode driver by a level shifter to control pulse width, pulse frequency, output polarity and output mode of current stimulation pulses via input control signals.

The neural signal detection module may use any combination of two or more stimulation electrode contacts as measuring electrode inputs to detect a nerve area potential or an electrocorticogram and feedback the measured signal to the multifunctional headgear 2 via the bidirectional radio frequency data communication module and the first coil 12. The inertial measurement processor monitors movement of the patient, such as walking and sleeping. Additionally, the inertial measurement processor may also detect cardiac rhythm changes in the patient, providing a possible tool for detection of the cardiac rhythm changes. The neural signal detection module combines any of two or more stimulation electrode contacts as measuring electrode inputs for detecting potential of the nerve area, and the measured signals are stored in internal memory of the implant as required or fed back to the multifunctional headgear 2 via the bidirectional radio frequency data communication module and the first coil 12; and a first Bluetooth transceiver is used for receiving commands from the patient remote controller 3, such as selecting a stimulation prescription, setting a stimulation intensity, and turning on/off stimulation, etc.

The present invention achieves a subminiature implantable stimulator design with a volume of approximately 4 ml using a highly integrated multifunction ASIC chip. The implant stimulator 1 includes 8 or more independent current pulse stimulation channels, two at least 4 channel electrode connectors 118 connected to two stimulation electrodes 13 including at least 4 stimulation contacts, a first rechargeable battery 114 and a first coil 12 that can be quickly charged. The craniotomy area required for implant surgery is limited to about 25 mm*25 mm, so as to allow the implant stimulator 1 to be implanted at an optimal implantation site of human skull according to the treatment requirements, and provide electrical stimulation for the treatment of various sites of cranial nerve, such as subthalamic nucleus, medial part of globus pallidus, hippocampus, etc. so as to treat diseases such as Parkinson's disease, epilepsy and depression.

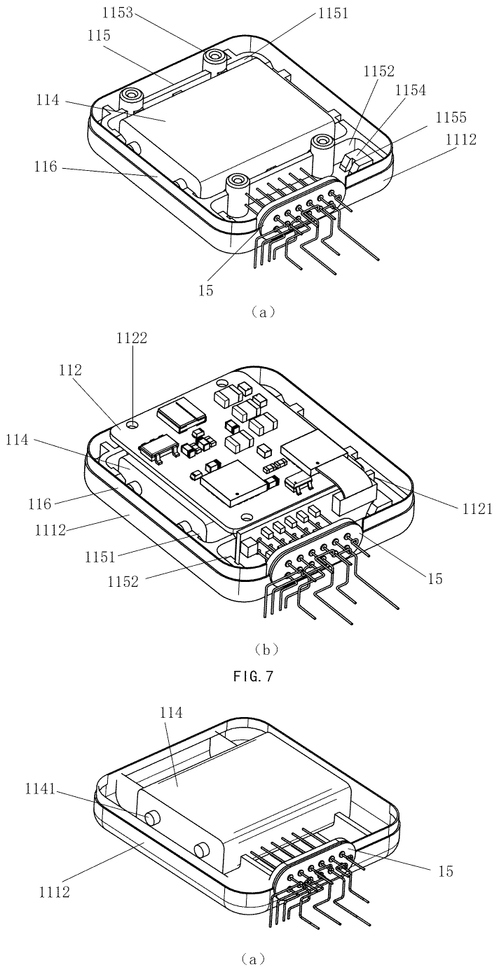

FIG. 7(a) is an internal assembly view of a metal shell according to a first embodiment of the present invention, and FIG. 7(b) is an internal assembly view of a circuit mainboard mounted in a metal shell according to a first embodiment of the present invention.

Referring to FIGS. 3b, 7(a) and 7(b), the metal shell 111 includes a first shell 1111 and a second shell 1112 which are tightly fitted, and in the first embodiment, a pressure-sensitive adhesive sheet 117, a frame 115, a first rechargeable battery 114, a rubber gasket 113 and a circuit mainboard 112 are successively provided between the first shell 1111 and the second shell 1112, and the frame 115 and the circuit mainboard 112 are tightly fitted. Further, a PCB mounting board 1121 is vertically connected to a side edge of the circuit mainboard 112 and is used for connecting a pin of the feedthrough connector 15 in the machine shell; the frame 115 is divided into a first space 1151 and a second space 1152; the first rechargeable battery 114 is placed in the first space 1151; the first rechargeable battery 114 can be provided in a rectangular parallelepiped shape; an end side of the second space 1152 is an open end and is matched with and mounted with the feedthrough connector 15; the second space 1152 is provided with a convex block 1154 along side walls of two sides; and a slot 1155 is provided on the convex block 1154. The two ends of the PCB mounting board 1121 are inserted and fixed in the slot 1155; the pin of the feedthrough connector 15 placed on the end side of the second space 1152 is connected to the mounting board 1121; a protective solder strip 116 is provided on the periphery of the frame 115; the protective solder strip 116 surrounds the periphery of the frame 115 and is fixed against the feedthrough connector 15 on the end side of the second space 1152; and correspondingly, an opening for placing the feedthrough connector 15 is also provided on the second shell 1112. In the present embodiment, the ASIC chip, the first microprocessor, the first Bluetooth transceiver and the inertial measurement processor are all arranged in the same side of the circuit mainboard 112, and the first rechargeable battery 114 is placed on the other side of the circuit mainboard 112, so as to reduce the space height as far as possible, so as to meet the requirements of not exceeding the thickness of the skull; the design is compact, so that the size of the skull is about 25*25 mm, so as to realize a minimal wound in an implantation surgery for a brain pacemaker. The thickness of the metal shell 111 can be controlled to about 6 mm. A plurality of fixed columns 1153 with threaded holes are provided on a side of the first space 1151 of the frame 115, a corresponding first through hole 1122 is arranged on the circuit mainboard 112, and after screws are used to pass through the first through hole 1122 and are fixed on the threaded holes on the fixed columns 1153, the circuit mainboard 112 is fixed on the frame 115, and the first charging battery 112 is limited in the first space 1151.

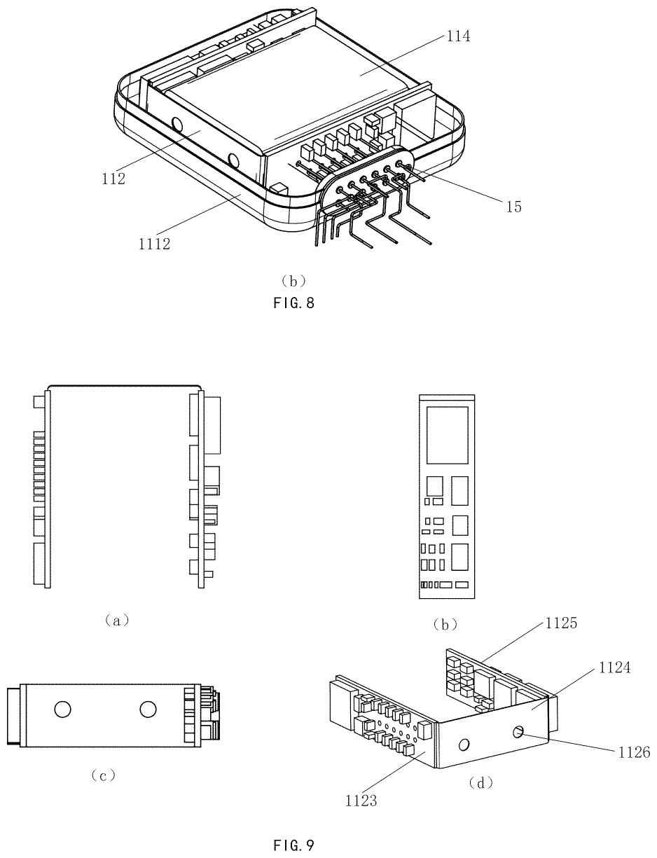

FIG. 8(a) is an internal assembly view of a metal shell according to a second embodiment of the present invention, and FIG. 8(b) is an internal assembly view of a circuit mainboard mounted in a metal shell according to a second embodiment of the present invention; and FIG. 9(a) and 9(d) are a top view, a side view, a front view and a solid diagram, respectively, of a circuit mainboard according to a second embodiment of the present invention.

Referring to FIG. 8(a) to 9(d), in a second embodiment, the circuit mainboard 112 is a rigid-flexible circuit mainboard which is bent to surround a first rechargeable battery 114, and specifically includes a first side 1123, a second side 1124 and a third side 1125, where the first side 1123 is connected to the pins of the feedthrough connector 15, the third side 1125 is provided with various components, the second side 1124 is provided with a second through hole 1126, and the protruding positive electrode/negative electrode 1141 at the end of the first rechargeable battery 114 is inserted into the second through hole 1126. This structure makes the volume of the implant stimulator 1 small, and this structure can realize that the total thickness of the implant stimulator 1 can meet the requirements for transcranial mounting under the condition that the battery thickness is close to 5 mm. Other structures are similar to those of the first embodiment.

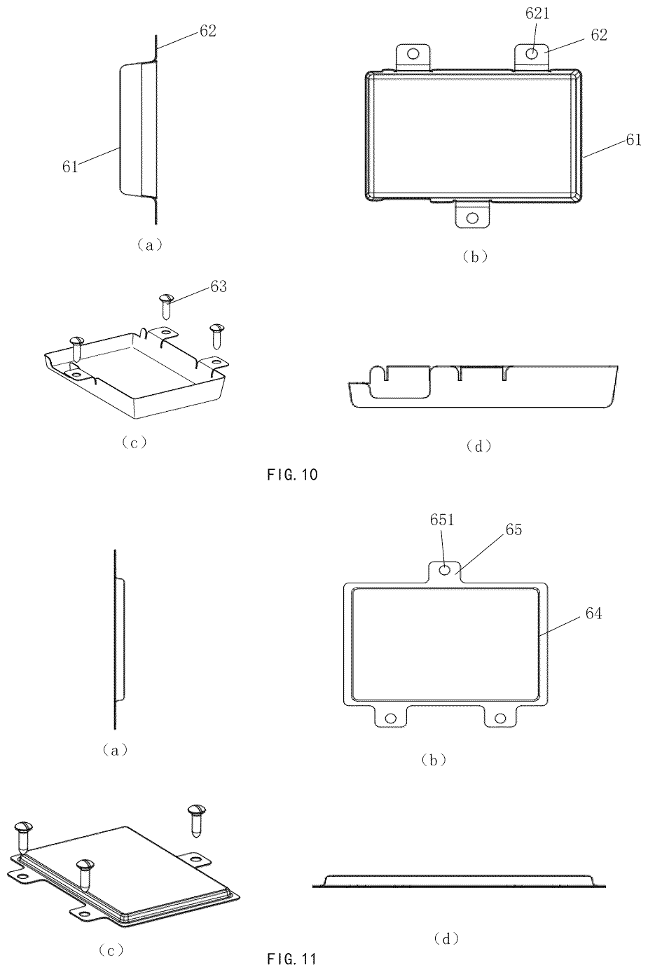

FIG. 10(a), FIG. 10(b), FIG. 10(c) and FIG. 10(d) are a side view, a top view, a solid diagram and a front view, respectively, of a base plate of an implant mounting bracket according to a first embodiment of the present invention; and FIG. 11(a), FIG. 11(b), FIG. 11(c) and FIG. 11(d) are a side view, a top view, a solid diagram and a front view, respectively, of an upper cover of an implant mounting bracket according to a first embodiment of the present invention.

Referring to FIG. 10(a)-11(d), in the first embodiment, the implant stimulator 1 further includes a mounting bracket 6, where the mounting bracket 6 includes a base plate 61, a cover plate 64 and a screw 63, the edges of the base plate 61 and the cover plate 64 are provided with hangers, and threaded holes are provided on the hangers, specifically, the edge of the base plate 61 is formed with a plurality of first hangers 62, preferably, the number of the first hangers 62 is three, and the first hangers are respectively arranged outside two long sides of the base plate 61, and a first threaded hole 621 is provided in each first hanger 62; the cover plate 64 is matched and buckled on the base plate 61, a plurality of second hangers 65 are formed on the edge of the cover plate 64, and preferably, the number of the second hangers 65 is three, and the second hangers are separated from the two long sides of the cover plate 64, and a second threaded hole 651 is provided in each of the second hangers 65. After mounting, the second hangers 65 and the first hangers 62 are provided in a staggered manner, one side of the mounting bracket 6 includes two first hangers 62 and one second hanger 65 provided between the two first hangers 62, and the other side of the mounting bracket 6 includes two second hangers 65 and the first hanger 62 provided between the two second hangers 65. The screws 63 are used to secure the base plate 61 and cover plate 64 to an open edge of the skull 5.

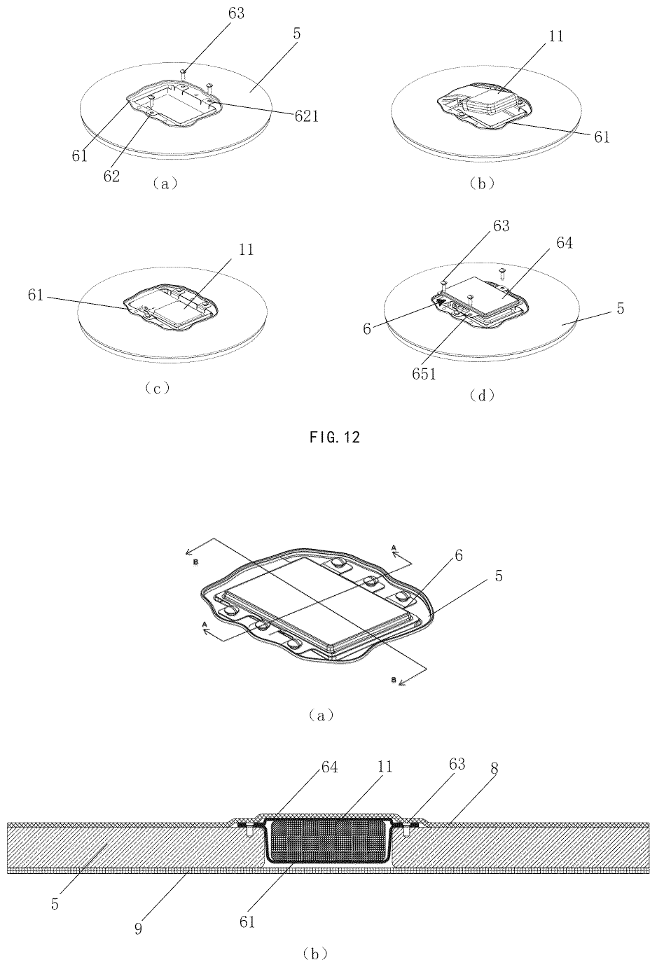

FIG. 12(a) is a schematic diagram showing a base plate of a mounting bracket on the skull according to a first embodiment of the present invention, FIG. 12(b) and FIG. 12(c) are schematic diagrams showing an implant stimulator inserted between a skull and a scalp and an implant main body placed in the base plate according to a first embodiment of the present invention, and FIG. 12(d) is a schematic diagram showing a cover plate covered on a base plate according to the first embodiment of the present invention.

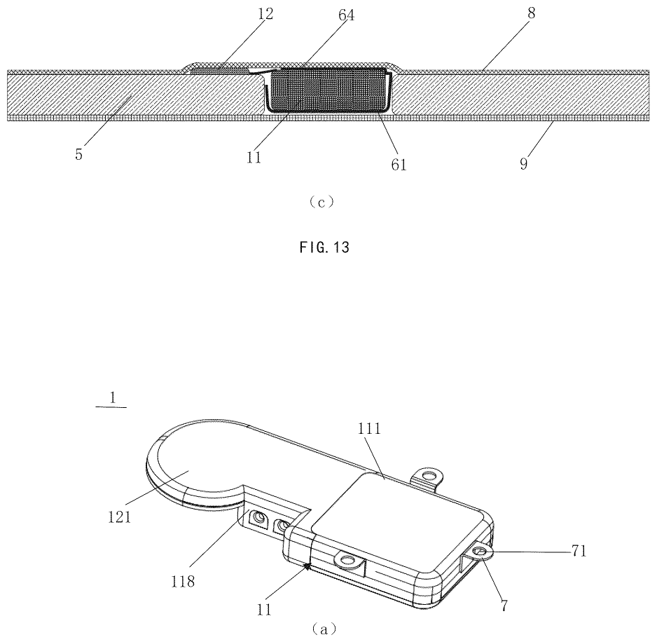

When the implant stimulator 1 is implanted into the skull, the process in which the implant stimulator 1 is mounted on the skull 5 via the mounting bracket 6 is as follows: Referring to FIG. 12(a), firstly, the base plate 61 is mounted on the skull 5 via a screw 63 through a first threaded hole 621; secondly, referring to FIG. 12(b) and FIG. 12(c), the implant main body 11 is placed on the base plate 61, and the first coil 12 is placed between the scalp 8 and the skull 5; as shown in FIG. 13(c), the thin flexible first coil 12 arranged outside the implant main body 11 can be directly placed between the skull 5 and the scalp 8, which not only facilitates mounting and saves the area of the skull opening, but also serves to fix the implant main body 11 mounted on the skull; then, referring to FIG. 12(d), a cover plate 64 is covered on the top of the implant main body 11, so that the implant main body 11 is enclosed in the chamber formed by the base plate 61 and the cover plate 64, and the cover plate 64 is mounted on the skull 5 through the second threaded hole 651 via the screw 63. The mounting bracket 6 is mounted as shown in FIG. 13(a) and 13(b). The surface of the implant main body 11 mounted with the cover plate 64 is substantially flush with the surface at the first coil 12.

The use of the mounting bracket 6 including the base plate 61 and the cover plate 64 as a fixing means for the implant stimulator 1 facilitates the surgery while preventing possible damage to the implant stimulator 1 when the skull hits a hard object.

Due to the ultra-thin structure of the implant stimulator 1, the total transcranial mounting thickness of the implant main body 11 including the mounting bracket 6 does not exceed 4 mm. The skull of a human body is typically from 6 to 10 mm, and the Chinese respectively have a relatively thin skull of from 6 to 8 mm, so the present invention provides that the implant stimulator 1 can be safely fixed to the skull 5 and placed on the cranial dura 9, achieving the following advantages: (1) It is convenient for the surgery, reduces surgical trauma, eliminates the burdens and risks of electrode extension, overcome the adverse events such as infection and electrode wire fracture that are easy to occur in clinical treatment, and improve the treatment effect. (2) Another advantage of implanting a brain pacemaker on the skull is that it reduces the number of surgical steps and time required for a clinician to make a tunnel and wire tunneling, and also reduces the risk of surgery because the patient has complicated structures near the head and neck, such as arteries, veins, and vagus nerves, and the risk of electrode wire tunneling is high. (3) Another advantage is that the replacement of the implant stimulator 1 and the removal procedure are greatly simplified, because a doctor only needs to remove the implant stimulator 1 on the skull, but does not need to pull out the electrode extension wire again, while the risk of pulling out the electrode wire is reduced, because the long-term implanted wire is easy to form connective tissue around the patient's neck, and the doctor may need to separate it before pulling out the stimulation electrode, with high surgical risk and great patient trauma.

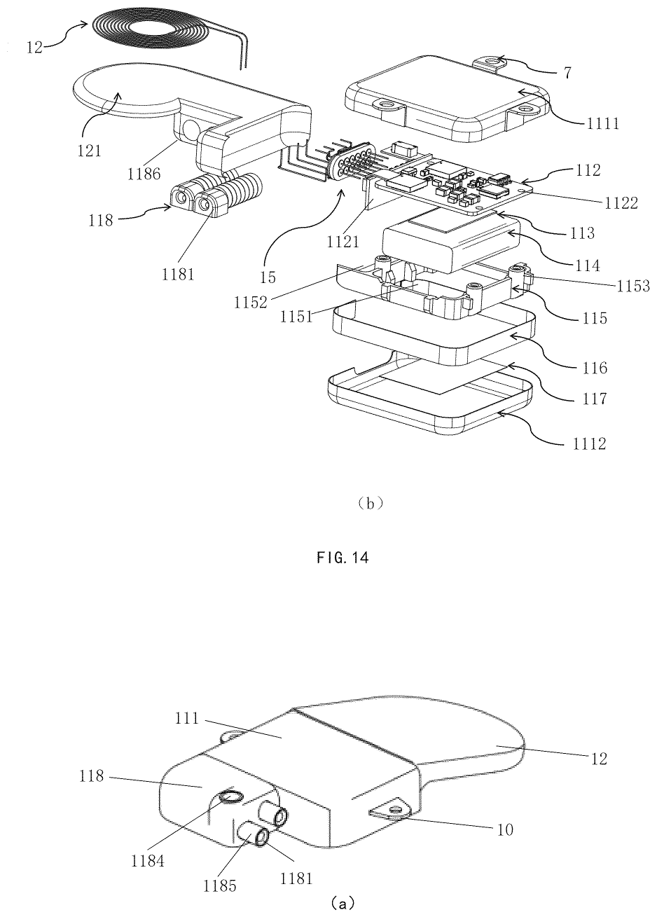

FIG. 14(a) is a schematic overall structural diagram showing an implant stimulator according to a third embodiment of the present invention, and FIG. 14(b) is an exploded schematic diagram showing an implant stimulator according to a third embodiment of the present invention.

Referring to FIG. 14(a) and 14(b), in the third embodiment, the first shell 1111 is provided with a plurality of first fixed hanging plates 7. Unlike the first and second embodiments, in the present embodiment, no mounting bracket 6 is required, which simplifies the structure. Specifically, a first fixed hanging plate 7 is provided on each of three sides of the first shell 1111, a third threaded hole 71 is provided on each first fixed hanging plate 7, the implant stimulator 1 is fixed on the skull 5 via a screw 63 extending through the third threaded hole 71, and the first fixed hanging plate 7 can be fixed on the first shell 1111 by means of welding.

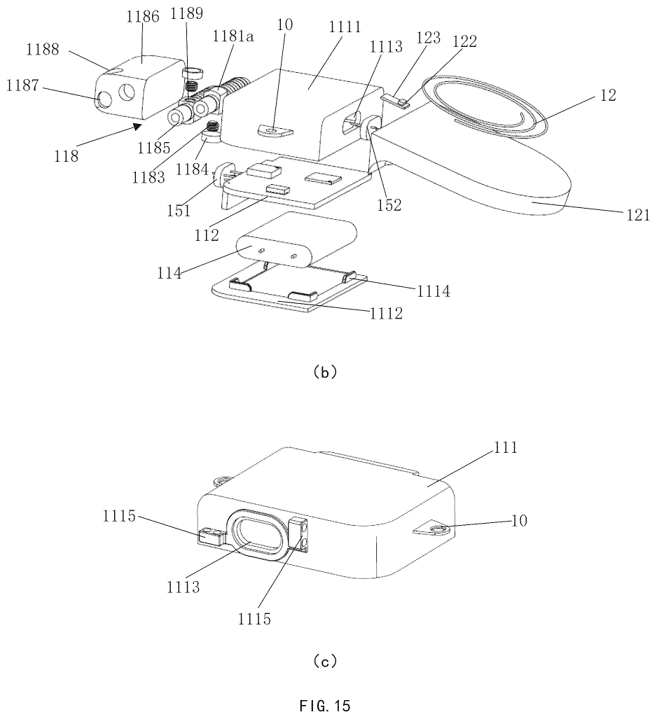

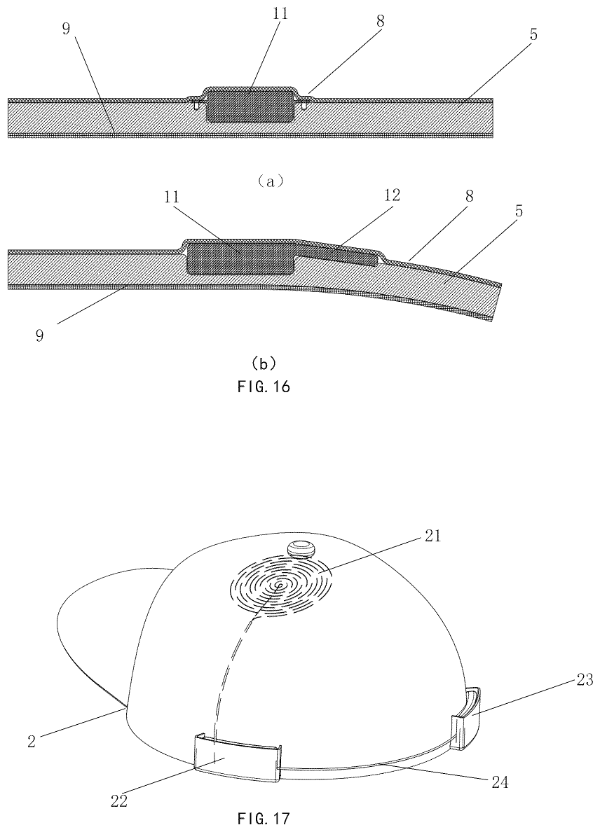

FIG. 15(a) is a schematic overall structural diagram showing an implant stimulator according to a fourth embodiment of the present invention; FIG. 15(b) is an exploded schematic diagram showing an implant stimulator according to a fourth embodiment of the present invention; FIG. 16(a) is a schematic cross-sectional transverse perspective view after mounting an implant stimulator according to a fourth embodiment of the present invention; and FIG. 16(b) is a schematic longitudinal perspective view after mounting of an implant stimulator according to a fourth embodiment of the present invention.

Referring to FIG. 15(a) and 15(b), in a fourth embodiment, in order to separate an antenna signal from a stimulation signal and avoid mutual interference, a first coil 12 and an electrode connector 118 are respectively provided on two sides of a metal shell 111, and the electrode connector 118 is encapsulated with epoxy resin or polyurethane (PU) and is connected to a current pulse output channel of a current pulse generator via pins of a feedthrough connector 151 so as to output a stimulation signal, where the number of the pins of the feedthrough connector 151 is eight; the first coil 12 is encapsulated with a flexible material such as silica gel or polyurethane (PU) and is respectively connected to a charging module, a data communication module and a first Bluetooth transceiver on the circuit mainboard 112 via pins of a feedthrough connector 152; further, as in the first embodiment, a Bluetooth communication component is provided in the protective sleeve 121, and the Bluetooth communication component includes a Bluetooth chip antenna 122 and a ceramic circuit board 123 carrying the Bluetooth chip antenna 122; in other embodiments, the Bluetooth communication component can also be a wire antenna wound according to a predetermined shape or a predetermined length; the Bluetooth communication component is encapsulated with epoxy resin or other insulating materials and then encapsulated with a flexible material such as silica gel or polyurethane (PU) together with the first coil 12; an output end of the first coil 12 is connected to the charging module and the data communication module on the circuit mainboard 112 via pins of the feedthrough connector 152; an output end of the Bluetooth communication component is connected to the first Bluetooth transceiver on the circuit mainboard 112 via the pins of the feedthrough connector 152; the number of the pins of the feedthrough connector 152 is four, two of the pins are connected to the output end of the first coil 12, and the other two pins are connected to the output end of the Bluetooth communication module.

The electrode connector 118 includes a connection socket 1181 and a connector shell 1186 arranged outside the connection socket 1181, where the connector shell 1186 is preferably formed by pouring when being encapsulated by epoxy resin; the connection socket 1181 includes a retaining member 1181a and an annular electrode contact 1182; a fourth through hole 1187 is provided on a front wall of the connector shell 1186; the fourth through hole 1187 allows the stimulation electrode 13 to be inserted into the connection socket 1181; the retaining member 1181a and the electrode contact 1182 are placed inside the connector shell 1186; the annular electrode contact 1182 is composed of a plurality of metal rings and metal coils located in the metal rings, an insulating ring is provided between two adjacent metal rings, a fifth through hole 1188 is provided on the upper and lower faces of the connector shell 1186, a retaining member 1181a is provided with a fourth threaded hole 1189 corresponding to the fifth through hole 1188, and after the screw 1183 extends through the fifth through hole 1188 and is screwed to the fourth threaded hole 1189, the stimulation electrode 13 is fastened in the connection socket 1181; a sealing member 1184 is inserted into the fifth through hole 1188 to perform sealing, and further, a metal ring and an insulating ring are bonded and fixed; an electrode protective sleeve 1185 is provided at the end of the retaining member 1181a; the electrode protective sleeve 1185 can protect the stimulation electrode 13; the sealing member 1184 and the electrode protective sleeve 1185 are both preferably made of silicone, and a preliminary point of silicone pre-fixing can be performed between the electrode protective sleeve 1185 and the retaining member 1181a, and then epoxy resin or polyurethane (PU) is encapsulated and fixed together. Further, the electrode connector 118 is directly adhered to the side wall of the metal shell 111, or a fixed block 1115 is provided on the side wall of the metal shell 111 connected with the electrode connector 118, and the electrode connector 118 is connected to the side wall of the metal shell 111 through the fixed block 1115 when it is encapsulated with epoxy resin or polyurethane (PU).

The present embodiment provides an implant stimulator 1 with a more reasonable arrangement of the electronic components and orientation of circuit wiring, so that an output signal of the implant stimulator 1 is more effective, and at the same time, the manufacturability of the structure is increased. Because of the ultra-small size, various process difficulties are exponentially increased when assembling the product, and the manufacturability and testability of the product are considered when designing, which is a prerequisite for ensuring the reliability of the product.

With continuing reference to FIG. 15(b), in the fourth embodiment, the metal shell 111 includes a first shell 1111 and a second shell 1112 which are tightly fitted, where the second shell 1112 is one bottom board, a protruding strip 1114 is provided on the bottom board, the first rechargeable battery 114 is placed within enclosure of the protruding strip 1114, and insulating materials are provided at the contact positions of the second shell 1112, the first rechargeable battery 114 and the circuit mainboard 112 for electrical isolation, for example: an electrically insulating material is interposed between the protruding strip 1114 and the first rechargeable battery 114. The circuit mainboard 112 is installed on top of the first rechargeable battery 114; the first shell 1111 is box shaped and is snap-fit onto the second shell 1112; a third through hole 1113 matched with the feedthrough connectors 151 and 152 is provided on a side wall of the first shell 1111 connected to the first coil 12 and the electrode connector 118; the feedthrough connectors 151 and 152 at two sides are mounted in the corresponding third through hole 1113; a second fixed hanging plate 10 is provided on the other two side walls of the first shell 1111, and the second fixed hanging plate 10 is used for fixed mounting on the skull 5.

Referring to FIG. 16(a) and 16(b), the implant stimulator 1 disclosed in the present embodiment is an implant method by which the implant main body 11 is implanted on the skull 5 without perforating the skull 5. This structure requires that the metal shell 111 of the implant main body 11 has an increased strength to be able to withstand the consequences of the implant main body 11 protruding out of the scalp, so that the thickness of the metal shell 111 is increased for this purpose. Generally, the shell thickness of the implant stimulator 1 is from 0.2 to 0.3 mm, the thickness of the second shell 1112 of the lower shell of the metal shell 111 in the present embodiment is from 0.4 to 0.6 mm, increased to 0.75 mm around, and the thickness of the first shell 1111 is increased to 0.5 mm, so as to ensure the strength requirement of the impact. Therefore, when the implant stimulator 1 provided in the present embodiment is implanted into the human body, the implant main body 11 is directly implanted on the skull, without perforating the skull, and the first coil 12 and the electrode connector 118 are separately installed on two sides of the metal shell 111, which is more beneficial to the transmission effectiveness and accuracy of the electronic signal.

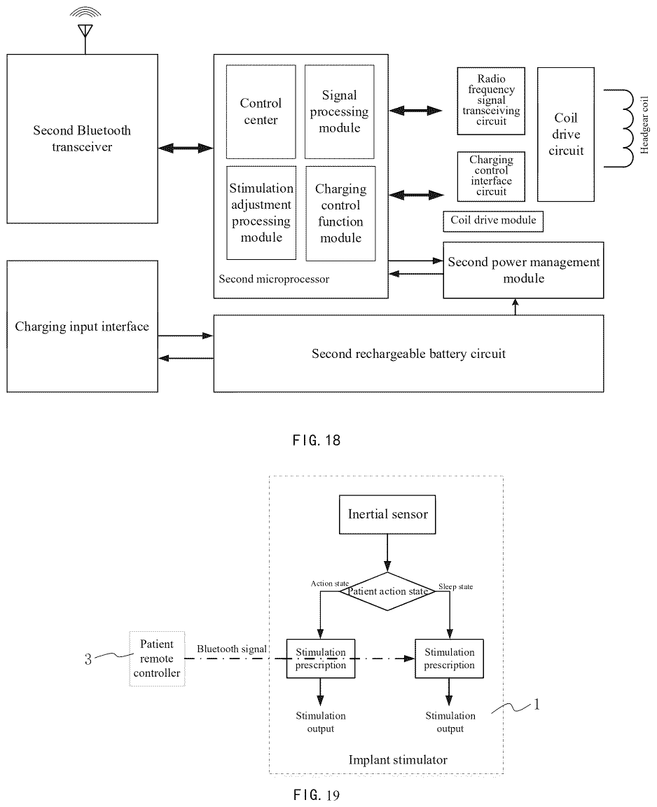

FIG. 17 is a schematic structural diagram showing a multifunctional headgear according to an embodiment of the present invention; and FIG. 18 is a schematic diagram showing a circuit of a multifunctional headgear according to an embodiment of the present invention.

With the features of the implant stimulator 1 mounted on the head and the first coil 12, the system is equipped with an important multifunctional headgear 2 to achieve an object of the present invention: 1) Realizing high-efficiency charging and high-reliability short-distance bidirectional data communication; 2) A design of the multifunctional headgear is intended to make the headgear conform to the head tightly and comfortably when a patient wears it, and the headgear does not require additional auxiliary fixing means such as bandage when operating with the implant stimulator 1, which does not affect the patient's free movement; 3) By using the neural signal and motion signal detection function of the implant stimulator 1 and through near-distance bidirectional data communication, a clinical data acquisition platform under the environment of real-time stimulation and free movement of the patient is provided; 4) With the neural signal and motion signal detection function of the implant stimulator 1, through close range bi-directional data communication, it is helpful to realize the recording, processing and analysis of feedback signal, so as to realize the closed-loop control function of brain stimulation in the working environment without affecting the battery power consumption of the implant.

Referring to FIG. 17, the multifunctional headgear 2 is provided with a second coil 21, a controller 22, and a second rechargeable battery 23, the controller 22 is connected with the second rechargeable battery 23 via a flexible cable 24, and the controller 22 is connected with the second coil 21 via the flexible cable 24, respectively, and the multifunctional headgear 2 is a hat conforming to the skull contour of a patient, and the hat can be designed in various styles, which is not particularly limited to the present invention. In a worn state of the multifunctional headgear 2, the second coil 21 covers the first coil 12, and the dimensions of the second coil 21 and the design of the controller 22 are such as to ensure that there is additional space for the movement of the headgear when it is worn. The second coil 21 is a flat coil spirally wound by a twisted cable or a single wire twisted by a plurality of insulated copper wires, the periphery of the second coil 21 is encapsulated by injection molding of silica gel or other flexible material and then sewed in an appropriate position in the fabric of the multifunctional headgear 2, the controller 22 is a thin rigid-flexible circuit mainboard encapsulated by epoxy resin or other insulating material and sewed in the fabric at one side edge of the multifunctional headgear 2, and the second rechargeable battery 23 is sewed at the other side edge of the headgear. The second rechargeable battery 23 is preferably a rechargeable lithium battery. The capacity of the second rechargeable battery 23 ensures that the built-in first rechargeable battery 114 of the implant stimulator 1 can be fully charged more than twice, or continuously operated for more than 12 hours in the closed-loop stimulation mode. The charging of the second rechargeable battery 23 may be achieved by a USB or a special interface through a charging module circuit. The total weight of the multifunctional headgear 2, including the second rechargeable battery 23, does not exceed 250 grams, so that the patient is not affected by the feeling of load when wearing the headgear.

Referring to FIG. 18, the controller 22 includes a second microprocessor, a second Bluetooth transceiver, a second power management module and a coil drive module, the second microprocessor includes a signal processing module, a stimulation adjustment processing module and a charging control functional module respectively used for processing and analyzing signals fed back by the implant stimulator, adjusting stimulation parameters according to the feedback signals and managing the charging process of the multifunctional headgear on the implant; the coil drive module includes a coil drive circuit, a radio frequency signal transceiving circuit and a coil induction charging control interface circuit; the second power management module includes a charging circuit of a second rechargeable battery, a charging input interface, a battery protection circuit and a voltage regulation circuit; the voltage regulation circuit is responsible for providing a controllable driving voltage for the coil drive circuit and a regulated voltage of other circuits; the charging input interface is responsible for charging the second rechargeable battery via an external charging power supply; and the battery protection circuit provides protection for the second rechargeable battery. The second Bluetooth transceiver provides a Bluetooth communication channel of the multifunctional headgear 2. The signal processing module is configured to process a signal fed back by the implant stimulator and extract useful information, and the stimulation adjustment processing module adjusts the stimulation parameters of the implant according to the feedback signal. Further, the coil drive module is provided with a security support circuit ensuring radio frequency charging of the implant stimulator. For an object of wearing for a long period of time without affecting the patient's free movement, design specifications of the coil drive module are high efficiency power output and maintaining surface temperature rise of the multifunctional headgear 2 within a safe range. Further, the controller is provided with a radio frequency signal strength detection circuit for determining and reminding whether the position of the multifunctional headgear is shifted.

Thus, the multifunctional headgear 2 achieves the following functions: (1) It acts as a charger for the implant stimulator 1: the first rechargeable battery 114 in the implant stimulator 1 is wirelessly charged through coil coupling, (2) Communication hub: it performs short-distance high-speed radio frequency data communication with the implant stimulator 1 through coil coupling, receives the feedback data from the implant stimulator 1 and sends a control command. At the same time, it communicates with the program controller 4 and the remote controller used by the patient via the Bluetooth channel, and the program controller 4 is provided with a surgery mode to allow data exchange with the implant stimulator 1 directly or via the multifunctional headgear 2 or the patient remote controller transfer during a surgery recovery stage of the patient. (3) Data processing and control center: the headgear records the data fed back by the implant stimulator 1, processes and analyzes the data fed back in a closed-loop mode, extracts the information related to treatment, and determines the real-time adjustment of stimulation parameters.

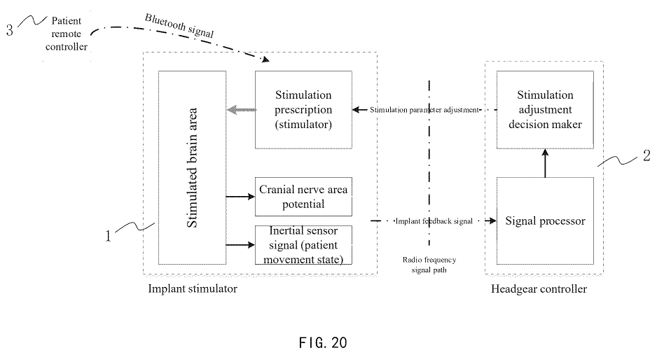

FIG. 19 is a schematic diagram showing an autonomic mode of a brain nerve electrical stimulation system according to an embodiment of the present invention; and FIG. 20 is a schematic diagram showing a closed-loop mode of a brain nerve electrical stimulation system according to an embodiment of the present invention.

The present embodiment provides a brain nerve electrical stimulation system provided with a dual operation mode, i.e., an implant stimulator autonomous stimulation mode and a system closed-loop mode.

In the autonomous stimulation mode, the open loop operation mode of the implant stimulator 1 is the same as that of the existing implant stimulator in clinical use. The operation flow is as shown in FIG. 19: the stimulation parameters and stimulation modes set by the implant stimulator 1 according to the stimulation prescription, including stimulation electrode contact combination, stimulation polarity, beat, pulse amplitude range, frequency, width, modulation mode, etc. There may be a plurality of stimulation prescriptions, the stimulation parameters are determined according to the stimulation effect within the stimulation security threshold in the initial debugging and testing stage after the stimulator implantation surgery, and the patient can select the stimulation prescription and adjust the stimulation intensity through the remote controller 3 according to the specific feeling and occasion requirements. The implant stimulator 1 can automatically change the stimulation prescription or adjust the stimulation intensity according to the posture and action of the patient detected by the inertial sensor, but the patient can also change the stimulation prescription through the remote controller 3.

In this autonomous stimulation mode, the multifunctional headgear 2 is required in two IPG operating states: implant stimulator charging and nerve potential recording. In addition, the system operating mode can only be switched by the multifunctional headgear.

The closed-loop mode of the system must be implemented jointly by the implant stimulator 1 and the multifunctional headgear 2. This system mode can be entered by the program controller 4 after the headgear 2 is worn and enters system pairing. The operation flow diagram for this mode is as shown in FIG. 20. In this mode, the stimulation prescription of the stimulator is still selected by the remote controller 3, but the neural signal detection function and the inertial measurement processor are activated; the implant stimulator 1 transmits the real-time nerve area potential signal and the patient action state signal to the headgear 2 via the radio frequency data channel; at the same time, the patient can also input some pre-determined state instructions via the remote controller 3, such as medication conditions, etc.; the multifunctional headgear 2 initiates the signal processing and stimulation decision function, determines new stimulation parameters according to the received feedback information and according to a pre-defined algorithm, and sends same to the implant stimulator 1 in time via the radio frequency data channel.

In the closed-loop system mode, the headgear can be allowed to simultaneously charge the implant stimulator battery or terminate the charge according to a command of the remote controller.

In the closed-loop system mode, if RF communication between the implant and the multifunctional headgear is interrupted, the implant stimulator automatically enters the autonomous stimulation mode to continue operation.

Although the present invention has been described with respect to one or more embodiments thereof, it will be understood by a person skilled in the art that the foregoing and various other changes, omissions and deviations in the form and detail thereof may be made without departing from the scope of the present invention.

What is claimed is:

1. A brain nerve electrical stimulation system with a sensing function, comprising a cranial-mounted implant stimulator with a stimulation electrode, a conveniently wearable multifunctional headgear, a patient remote controller and a clinician stimulation program controller; wherein
the implant stimulator comprises an implant main body and a first coil, the implant main body comprises an electronics enclosure sealed by a metal shell and an electrode connector encapsulated by a non-metal material, the first coil is installed outside the metal shell, a first power supply and a circuit mainboard are arranged inside the electronics enclosure, and a current pulse generator is arranged on the circuit mainboard; a current pulse output channels of the current pulse generator are connected to the stimulation electrode by means of the electrode connector to realize electrical stimulation function; the metal shell serves as a return electrode for current pulses;
the multifunctional headgear comprises a second coil, a second power supply and a controller, wherein the second coil, the second power supply and the controller are all sewn into a headgear fabric to be comfortably worn by the patient, and when the headgear is worn by the patient, the second coil and the first coil charge the first power supply through inductive coupling and perform radio frequency data communication as required;
the clinician stimulation program controller communicates with the multifunctional headgear via Bluetooth, setting up stimulation prescriptions for the implant stimulator, detecting and analyzing the operating condition of the implant stimulator, and exchanging data with a cloud server; the stimulation prescription comprises stimulating electrode contacts, stimulation mode, pulse width, pulse frequency, or/and, adjustable pulse amplitude range;
the patient remote controller is used by the patient to control over the implant stimulator, including starting or ending stimulation, adjusting stimulation intensity, inputting medication state, or/and starting or ending a charging process.

2. The brain nerve electrical stimulation system according to claim 1, wherein the 2n independent current pulse generators are arranged on the circuit mainboard to constitute 2n independently controllable current pulse stimulation channels, 2n annular electrode contacts are connected to the stimulation channels through the electrode connector, each of the current pulse stimulation channels of the stimulator in the hermetic electronics enclosure is connected to a corresponding contact of the electrode connector via pins of a feedthrough connector, and the electrode connector constitutes two n-channel connection sockets to be connected to all electrode wires of the two stimulation electrodes, respectively, each stimulation electrode containing n stimulation contacts, wherein n is a positive integer no less than 4.

3. The brain nerve electrical stimulation system according to claim 1, wherein a conductor of the first coil is a cable formed by twisting a plurality of pure gold wires, wherein the first coil is a flat coil with a protective sleeve and is spirally and horizontally wound with the cable and injection-molded from a biocompatible flexible material and has a thickness of less than 3 mm, and is integrally encapsulated with the metal shell by the flexible material; an output end of the first coil is connected to a charging module and a data communication module on the circuit mainboard in the hermetic electronics enclosure via the pins of the feedthrough connector, and when implanted into the human body, the first coil can be directly placed between the scalp and the skull, without cutting the skull open, and serves to fix the implant main body.

4. The brain nerve electrical stimulation system according to claim 3, wherein the first coil further comprises a Bluetooth communication component in the protective sleeve, the Bluetooth communication component is a Bluetooth chip antenna and a ceramic circuit board component carrying the Bluetooth chip antenna, or the Bluetooth communication component is a wire antenna wound in a predetermined shape or a predetermined length, the Bluetooth communication component is encapsulated with an insulating material and then encapsulated with the flexible material together with the first coil, and is connected to the first Bluetooth transceiver of the circuit mainboard via the pins of the feedthrough connector.

5. The brain nerve electrical stimulation system according to claim 4, wherein the first coil and the electrode connector are provided in the same side of the metal shell, the electrode connector and the protective sleeve of the first coil are integrally formed and then fixed on a side wall of the metal shell, and an output end of the first coil is connected to the charging module and the data communication module on the circuit mainboard via the pins of the feedthrough connector.

6. The brain nerve electrical stimulation system according to claim 4, wherein the first coil and the electrode connector are separately installed on two sides of the metal shell, the electrode connector is fixed on one side wall of the metal shell, the protective sleeve of the first coil is fixed on the other side wall of the metal shell, and when the implant stimulator is implanted into the human body, the implant main body is directly implanted on the skull without perforating the skull.

7. The brain nerve electrical stimulation system according to claim 6, wherein the first power supply is a first rechargeable battery, the metal shell comprises a first shell and a second shell which are tightly fitted together, the second shell is one bottom board, a protruding strip is provided on the bottom board, the first rechargeable battery is placed within enclosure of the protruding strip, the circuit mainboard is installed on top of the first rechargeable battery, and insulating material is provided at the contact places between the bottom board, the first rechargeable battery and the circuit mainboard for electrical isolation; the first shell is boxshape, and is snap-fit onto the second shell; a through hole matching the feedthrough connector is provided on the side wall of the second shell connected to the first coil and the electrode connector; the feedthrough connectors on two sides are respectively mounted in the corresponding through holes, and second fixed hanging plates are respectively provided on the other two side walls of the second shell.

8. The brain nerve electrical stimulation system according to claim 1, wherein the first power supply is a first rechargeable battery, an ASIC chip, a first microprocessor, a first Bluetooth transceiver and an inertial measurement processor are arranged on the circuit mainboard, the current pulse generator is provided in the ASIC chip, and the ASIC chip further comprises a neural signal detection module, a wireless charging management module, a bidirectional radio frequency data communication module and a first power management module, the first Bluetooth transceiver, the inertial measurement processor and the current pulse generator, the neural signal detection module and the bidirectional radio frequency data communication module are all connected to the first microprocessor interface; the first microprocessor controls input electrode selection, signal gain and frequency response range of the neural signal detection module, charging current and charging flow output by the wireless charging management module, and supply voltage, stimulation current, pulse width and pulse frequency of the current pulse generator. neural

9. The brain nerve electrical stimulation system according to claim 8, wherein the number of stimulation electrodes is two, and the stimulation electrodes can be used for stimulating areas of two brain hemispheres respectively, each electrode containing at least four stimulation contacts; the neural signal detection module combines any of two or more stimulation electrode contacts as measuring electrode inputs for detecting potential of the nerve area, and the measured signals are stored in internal memory of the implant as required or fed back to the multifunctional headgear via the bidirectional radio frequency data communication module and the first coil.

10. The brain nerve electrical stimulation system according to claim 8, wherein the inertial measurement processor detects patient motion and cardiac rhythm changes and feeds back the measured signal to the multifunctional headgear via the bidirectional radio frequency data communication module and the first coil.

11. The brain nerve electrical stimulation system according to claim 1, characterized by further comprising a mounting bracket comprising a base plate, a cover plate and screws, lugs are provided at edges of the base plate and the cover plate, wherein threaded holes are provided on the lugs, the screws can fix the base plate to an edge of an opening of the skull via the threaded holes, the implant main body is placed on the base plate, the cover plate covers the top of the implant main body, and the screws can fix the cover plate to the edge of the opening of the skull via the threaded holes on the cover plate, a surface of the implant main body with the cover plate mounted is substantially flush with a surface at the first coil.

12. The brain nerve electrical stimulation system according to claim 7, wherein the first shell is provided with a plurality of first fixed hanging plates, and the first fixed hanging plates are provided with third threaded holes.

13. The brain nerve electrical stimulation system according to claim 1, wherein the second coil is a flat coil spirally wound from a twisted cable twisted from a plurality of insulated wires, or from a single wire, the periphery of the second coil being injection molded from a flexible material, encapsulated and then sewn in place within the fabric of the multifunctional headgear, the multifunctional headgear being a cap conforming to the skull contour of a patient, the multifunctional headgear being worn with the second coil covering the first coil.

14. The brain nerve electrical stimulation system according to claim 1, wherein the second power supply is a second rechargeable battery, and the controller is connected to the second rechargeable battery and the controller is connected to the second coil via a flexible cable, respectively.

15. The brain nerve electrical stimulation system according to claim 14, wherein the controller comprises a second microprocessor, a second Bluetooth transceiver, a second power management module and a coil drive module, the second microprocessor comprises a signal processing module, a stimulation adjustment processing module and a charging control firmware functional module respectively used for processing and analyzing signals fed back by the implant stimulator, adjusting stimulation parameters according to the feedback signals and managing the charging process of the multifunctional headgear on the implant; the coil drive module comprises a coil drive circuit, a radio frequency signal transceiver circuit and a coil induction charging control interface circuit; the second power management module comprises a charging circuit of a second rechargeable battery, a charging input interface, a battery protection circuit and a voltage regulation circuit; and the second Bluetooth transceiver provides a Bluetooth communication channel of a multifunctional headgear.

16. The brain nerve electrical stimulation system according to claim 14, wherein the second coil is sewn into an insulating cap fabric corresponding to the first coil position of the implant, the controller is a thin rigid-flexible circuit mainboard encapsulated by epoxy resin and sewn into the fabric of one side edge of the multifunctional headgear, and the second rechargeable battery is sewn into the other side edge of the multifunctional headgear.

17. The brain nerve electrical stimulation system according to claim 1, wherein the controller is provided with a radio frequency signal strength detection circuit for determining and reminding whether the position of the multifunctional headgear is shifted.
